(19) United States
(12) Patent Application Publication
(10) Pub. No.: US 2009/0130687 A1
(43) Pub. Date: May 21, 2009

(54) FORMULATIONS AND METHOD ISOLATING NUCLEIC ACIDS FROM ARBITRARY COMPLEX STARTING MATERIALS AND SUBSEQUENT COMPLEX GENETIC MATERIALS

(76) Inventors: Peter Bendzko, Berlin (DE); Hans Joos, Berlin (DE)

Correspondence Address: BUCHANAN INGERSOLL & ROONEY PC, P.O. BOX 1404, ALEXANDRIA, VA, 22313-1404, US

(21) Appl. No.: 12/297,536

(22) PCT Filed: Apr. 18, 2007
(86) PCT No.: PCT/DE2007/000689

§ 371 Date: Oct. 17, 2008

(30) Foreign Application Priority Data
Apr. 25, 2006 (DE) 10 2006 019 650.3

(51) Int. Cl. C12Q 1/68; C07H 1/06; C12N 9/14; G01N 31/00; C07H 1/08

(52) U.S. Cl. 435/6; 536/127; 435/195; 536/128; 436/17

(57) ABSTRACT

The object of the invention is formulations and methods without chaotropic components for the isolation of nucleic acids with binding to a solid phase, in particular of DNA, from arbitrary complex starting materials containing a lysis/binding buffer system manifesting at least one anti-chaotropic salt component, the concentration of the anti-chaotropic salt components being between 0.001 mM and 0.1 M, preferably 0.1 mM, and further a solid phase and washing and elution buffers which are known per se.

The lysis/binding buffer system can exist as an aqueous solution or as a solid formulation in ready-to-use reaction vessels. As a solid phase, all carrier materials applied for isolation by means of chaotropic reagents can function, preferably glass fibre fleeces, glass membranes, silicone carriers, ceramics, zeoliths or materials possessing negatively functionalised surfaces or manifesting chemically modified surfaces which can be converted to a negative charging potential.

The object of the invention is further a method for the isolation of nucleic acids, in particular of DNA, from arbitrary complex starting materials making use of the formulations according to the invention, characterised by lysis of the starting material, binding of the nucleic acids to a carrier material, washing of the nucleic acids bound to the carrier and elution of the nucleic acids.

The object of the invention is formulations without chaotropic components for the isolation of nucleic acids with binding to a solid phase, in particular of DNA, from arbitrary complex starting materials and quantities containing a lysis/binding buffer system manifesting at least one anti-chaotropic salt component, one solid phase and a washing and elution buffer which is known per se. The lysis/binding buffer system can be available as an aqueous solution or as a solid formulation in reaction vessels ready for use. As a solid phase, all carrier materials applied for isolation by means of chaotropic reagents can function, preferably glass fibre fleeces, glass membranes, silicone carriers, ceramics, zeoliths or materials possessing negatively functionalised surfaces or manifesting chemically modified surfaces which can be converted to a negative charging potential.

The object of the invention is further a method for the isolation of nucleic acids, in particular of DNA, from arbitrary complex starting materials making use of the formulations according to the invention, marked by lysis of the starting material, binding of the nucleic acids to a carrier material, washing of the nucleic acids bound to the carrier and elution of the nucleic acids, in which the subsequent amplification of selected sequence sections and a subsequent analysis of the reproduced gene section can be carried out in one and the same reaction cavity if need be. The fields of application of the method are all laboratories concerning themselves with DNA isolations, such as forensic medicine, foodstuffs diagnostics, medical diagnostics, molecular biology, biochemistry, genetic engineering and all other neighbouring fields.

Under classical conditions, isolation of DNA from cells and tissues is done by the starting materials containing nucleic acids being dissolved under highly denaturising and reducing conditions, partly also with use of protein-decomposing enzymes, the resultant nucleic acid fractions being purified via phenol/chloroform extraction steps and the nucleic acids being obtained from the aqueous phase by means of dialysis or ethanol precipitation (Sambrook, J., Fritsch, E. F. and Maniatis, T., 1989, CSH, “Molecular Cloning”).

These “classical methods” for the isolation of nucleic acids from cells and in particular from tissues are very time-consuming (sometimes more than 48 h), demand considerable amounts of apparatus and additionally are also not feasible under field conditions. Also, such methods are an unsuitably large health risk as a result of chemicals used such as phenol and chloroform.

Various alternative methods for isolation of nucleic acids from various biological starting materials enable circumvention of the time-consuming, health-endangering phenol/chloroform extraction of nucleic acids and achieving a reduction of the time needed.

All these methods are based on a method developed and first described by Vogelstein and Gillespie (Proc. Natl. Acad. Sci. USA, 1979, 76, 615-619) for preparative and analytical purifying of DNA fragments from agarose gels. The method combines the dissolution of the agarose containing the DNA bands to be dissolved in a saturated solution of a chaotropic salt (NaJ) with a binding of the DNA to glass particles. The DNA fixed to the glass particles is then washed with a washing solution (20 mM Tris HCl [pH 7.2]; 200 mM NaCl; 2 mM EDTA; 50% v/v ethanol) and then removed from the carrier particles.

This method has been given a series of modifications in the meantime and is currently used for various methods of extraction and purifying of nucleic acids of varying origins (Marko, M. A., Chipperfield, R. and Birnboim, H. G., 1982, Anal. Biochem., 121, 382-387).

In addition, there are also a variety of reagent systems all over the world, above all for purifying of DNA fragments from agarose gels and for the isolation of plasmid DNA from bacterial lysates, but also of the isolation of longer-chained nucleic acids (genomic DNA, cellular total RNA) from blood, tissues or also cell cultures.

All these commercially available kits are based on the very well known principle of binding of nucleic acids to mineral carriers in the presence of solutions of differing chaotropic salts and use suspensions of finely ground glass powders (e.g. Glasmilk , BIO 101, La Jolla, Calif.), diatomaceous earths (firm of Sigma) or also silica gels (Diagen, DE 41 39 664 A1) as carrier materials.

A method for the isolation of nucleic acids practicable for a variety of various applications has been shown in U.S. Pat. No. 5,234,809 (Boom), in which a method for the isolation of nucleic acids from starting materials containing nucleic acid by incubation of the starting material with a chaotropic buffer and a solid phase binding the DNA is described. The chaotropic buffers achieve both the lysis of the starting material as well as the binding of the nucleic acids to the solid phase. The method is well suited to isolating nucleic acids from small sample quantities and specifically has its practical application in the area of isolation of viral nucleic acids.

Specific modifications of these methods are concerned with the use of new kinds of carrier materials, which show applicative benefits for certain questions (Invitek GmbH WO-A 95/34569).

Decisive disadvantages of methods of isolation of nucleic acids from complex starting materials on the basis of incubation of the starting material with a chaotropic buffer and a solid phase are, inter alia, the fact that the cell dissolutions to be achieved with the chaotropic buffer cannot be used for all materials and also only function extremely inefficiently and with great consumption of time for major quantities of starting materials Over and above this, mechanical homogenisation methods are necessary if, for example, DNA is to be isolated from tissue samples. Further, varying concentrations of varying chaotropic buffers have to be used for varying questions. Thus, the method is by no means suited for universal use.

Problems caused by a possibly difficult lysis of the starting material can be solved by a series of commercially available products for nucleic acid isolation (specifically for the isolation of genomic DNA from complex starting materials), but they have the great disadvantage that it is then no longer a question of a classical single-tube method which marks the method pursuant to the U.S. patent, as the lysis of the starting material is done in a customary buffer making use of a proteolytic enzyme. The chaotropic ions necessary for the subsequent binding of the nucleic acids to, for example, centrifugation membranes must be added to the lysis mixture separately after the lysis has taken place. But they cannot be a component part of the lysis buffer, as the protein-destroying function of chaotropic salts is known and would naturally also immediately destroy the proteolytic enzyme necessary for an efficient lysis.

Despite a series of disadvantages, the methods of nucleic acid isolation making use of chaotropic salts have therefore asserted themselves world-wide and are used in millions of cases by means of commercially available products. These systems are extremely simple in their implementation and always work according to the principle of the lysis of the starting material, the subsequent binding of the nucleic acid to the solid phase of a glass or silicon membrane located in a centrifugation column on a carrier suspension, washing of the bound nucleic acids and the subsequent elution of the nucleic acids with a buffer of a low ion strength.

All these systems are based on the binding of the nucleic acids to the carrier surfaces in question in the presence of chaotropic salts, i.e. at least one buffer solution contains a chaotropic salt as the main component. This can possibly affect the lysis buffer or, in the case of systems including proteolytic enzymes, a necessary binding buffer which is added following the lysis of the starting material.

The basis of chaotropic salts is the series of Hofmeister for precipitation of negatively charged, neutral or basic protein solutions. The chaotropic salts are characterised by the fact that they denaturise proteins, increase the solubility of non-polar substances in water and destroy hydrophobic interactions. According to the state of the art, precisely these properties cause the superior structure of the aqueous milieu in order to bring about the binding of the nucleic acids to selected solid phases in this way, even with buffer systems of chaotropic salts. The best known representatives for nucleic acid isolation are sodium perchlorate, sodium iodide, potassium iodide, guanidine isothiocyanate and guanidine hydrochloride. However, they are on the one hand cost-intensive and on the other hand partly toxic or corrosive.

On this state of the art, there are now a very large number of patent applications and granted patents, although it is always a question of variants of the method, e.g. the use of new carrier materials or more efficient washing buffers etc., the basic principle always being the use of chaotropic salts for the binding to a solid phase made of silica materials.

Later, it was seen (EP 1 135 479, owner; InViTek Gesellschaft für Biotechnologie & Biodesign mbH), that a variety of quite differing salts are sufficient as components of lysis/binding buffer systems, possibly already customary per se, for the binding of nucleic acids to classical carrier materials on the basis of glass or silica. The best results in this context were achieved with salts which manifest absolutely opposite effects with regard to the chaotropic salts used for nucleic acid binding up to now, according to their chemical/physical characteristics, salts which thus can be termed anti-chaotropic. For example, at least the same quantitative and qualitative results were achieved with lysis/binding buffers, the main components of which were, for example, ammonium salts instead of chaotropic salts (commercial extraction kits) in the extraction of genomic DNA from various complex starting materials (e.g. blood, tissue, plants), with a constancy of the other reaction components, carrier materials customary up to now and also with a completely identical sequence of the reaction. This means that with a salt which does not denaturise proteins, but stabilises them, which does not increase, but reduces the solubility of non-polar substances in water and which does not destroy, but reinforces hydrophobic interactions, it is equally possible to isolate, purify and feed nucleic acids, also from complex starting materials, to the applications which are customary per se. Anti-chaotropic components in the present context are ammonium, caesium, sodium and/or potassium salts, preferably ammonium chloride. According to EP 1 135 479, these anti-chaotropic salt components are used in ion strengths from 0.1 M to 8 M.

Completely surprisingly, it was now seen that, even in distinctly lower concentrations of the anti-chaotropic salt components, the required effect can be achieved. The present invention is accordingly concerned with formulations and methods without chaotropic components for the isolation of nucleic acids with binding to a solid phase, in particular of DNA from arbitrary complex starting materials containing a lysis/binding buffer system, manifesting at least one anti-chaotropic salt component, with the concentration of the anti-chaotropic salt component being between 0.001 mM and 0.1 M, preferably 0.1 mM, and further a solid phase and washing and elusion buffers which are known per se.

The lysis/binding buffer system further manifests detergents which are known per se and if applicable additives, e-g. Tris-HCl, EDTA, polyvinylpyrrolidone, CTAB, TritonX-100, N-lauryl-sarcosine, sodium citrate, DTT, SDS and/or Tween. In a preferred embodiment, the lysis/binding buffer system contains an alcohol for binding to the solid phase, e.g. ethanol and isopropyl alcohol and if applicable enzymes, preferably protein-decomposing enzymes, e.g. a proteinase.

Thus, via the use of new compositions of lysis/binding buffers on the basis of anti-chaotropic salts in a very low concentration for the isolation of nucleic acid, specifically for the isolation of genomic DNA, on the basis of the binding of the nucleic acids to the various solid phases from silica or glass materials, which are customary per se, the invention enables the use of an alternative chemistry as an essential component of corresponding test kits (formulations).

The method according to the invention, including anti-chaotropic salts, follows the sequences of methods known from practical laboratory routines for the isolation of nucleic acids and is characterised by:
1. lysis of the starting material
2. binding of the nucleic acids to a solid phase
(centrifugation column or suspension)
3. washing of the bound nucleic acids
4. elution of the nucleic acids with a low-salt buffer which is known per se.

The invention enables a highly efficient and quick isolation of nucleic acids, in particular genomic DNA from any arbitrary and also possibly complex starting material. The anti-chaotropic ions necessary for the binding can be components of the lysis/binding buffer, even if proteolytic enzymes are involved. The method according to the invention is thus simple to handle and can be used universally.

The isolation of nucleic acids, in particular of DNA, from arbitrary starting materials is implemented by the incubation of the starting material containing the nucleic acid without use of chaotropic substances, which are put into contact with
the lysis/binding buffer system, which entails an aqueous solution manifesting at least one anti-chaotropic salt component, at least one detergent, possibly additives and possibly an enzyme,
and an arbitrary solid phase, preferably glass fibre fleece, glass membranes, glasses, zeoliths, ceramic as well as other silicon carriers,
   by which the lysis of the starting material and the subsequent binding of the DNA to the solid phase takes place. After this, the bound nucleic acid is washed according to methods known per se and the DNA dissolved from the solid phase.

In certain extraction protocols, the lysis mixture can possibly be provided with an additional detergent, an alcohol or a detergent/alcohol mixture.

Preferred starting materials are compact plant materials such as fruits, seeds, leaves, needles etch, clinically relevant samples such as full blood, tissue, micro-bioptates, paraffinised materials, ercp samples, swab material from smears, foodstuffs such as fish, cooked meats, preserves, milk, forensic samples such as hair roots, cigarette ends, blood traces and other samples containing DNA.

Preferred ions within the meaning of the invention are the anti-chaotropic ammonium ions shown in the Hofmeister series, caesium ions as well as potassium and sodium ions or combinations of the said ions, preferably ammonium chloride.

As a result of the use of the anti-chaotropic salts, which have a protein-stabilising effect, proteolytic enzymes such as proteinase K, can also be added as essential components of a lysis buffer in a preferred embodiment of the invention to support the lysis process and to make it effective.

Buffer systems of the state of the art with the chaotropic salts known per se possibly do not contain any proteolytic enzymes at the necessary high ion strengths as generally demanded for a quantitative isolation of nucleic acids. Thus, they must always be added subsequently for the binding of the nucleic acids to the solid phases.

Anionic, cationic or neutral detergents such as SDS, Triton X-100, Tween or CTAB are preferably used in the lysis buffers/binding buffers according to the invention.

After the lysis of the starting material, the suspension is possibly separated from components not yet completely lysed with a short centrifugation step and directly incubated with the DNA-binding material or, as already described, incubated with the solid phase following addition of an additional detergent, an alcohol or a detergent/alcohol mixture. If necessary, there are additionally low concentrations (<50 mM) of EDTA and/or Tris-HCl in the lysis buffer system. For the isolation of DNA from very highly contaminated starting materials, there is also preferably the addition of 2-4% polyvinylpyrrolidone or other known substances to the buffer system for selective binding of inhibitory components.

As binding materials for the DNA to be isolated, for example, commercially available glass fibre fleeces in centrifugation columns, silicon compounds such as SiO₂ of varying particle sizes have outstandingly proven their worth. In this way, all the materials used for the isolation of nucleic acids by means of chaotropic buffers can also be used.

After incubation with the material binding the DNA, the lysate is separated from the binding material by a short centrifugation step. After this, there is washing in a way known per se with a washing buffer, e.g. entailing at least 50% ethanol and if need be a low salt concentration, e.g. NaCl, the carrier material is dried and the bound DNA eluted by means of a low-salt buffer known per se (Tris-HCl; TE; water) and at a preferred temperature of 50-70° C.

A further embodiment of the invention comprises the addition of proteolytic enzymes, preferably proteinases, e.g. proteinase K, for lysis of starting materials which are hard to dissolve, e.g. compact tissue samples, hair roots, or for optimisation of the lysis efficiency and to reduce the necessary lysis times.

The invention thus enables methods for universal use for the isolation of nucleic acids, in particular DNA, from all starting materials containing DNA and also from arbitrary quantities of varying starting materials on new combinations of anti-chaotropic salts as essential components of lysis buffer mixtures, in which context all the carrier materials and their embodiments used up to now can be used equally as efficiently as the directives of isolation practised tip to now are identically usable.

In its most general embodiment, a nucleic acid extraction can be done by means of the method according to the invention from complex starting materials selected and corresponding to the state of the art for a DNA extraction, that is to say that the new universal buffer system permits successful, extremely simple and very fast highly efficient lysis and subsequent binding of nucleic acid to a mineral carrier of compact plant material (such as fruits, seeds, leaves, needles etc.), from clinically relevant samples (such as full blood, tissue, micro-bioptates, paraffinised materials, ercp samples, swab material from smears), from foodstuffs (such as fish, cooked meats, preserves, milk), from forensic samples (z, such as hair roots, cigarette ends, blood traces) and also from other starting materials.

A further advantage of the method is the fact that the isolation of DNA can be done highly efficiently both from extremely slight starting materials (e.g. isolation of DNA from 1 μl of full blood; hair root, micro-biopsy <1 mg) and also from very large quantities of starting materials such as 50 ml of full blood; 1 g of tissue material, <1 g of plant material.

Alongside a highly general embodiment, optimisations of the extraction method relative to specific applications even permit an almost quantitative isolation of the quantities of DNA contained in the initial sample.

The method according to the invention is also outstandingly suited to the design of automation-capable systems in which price/preparation is known to be a decisive selection criterion.

The formulations according to the invention surprisingly permit access to further highly interesting and new kinds of applications in the field of isolation of nucleic acids and diagnostics.

In a further embodiment of the invention, the existing new lysis/binding buffer systems manifesting at least one anti-chaotropic salt component are in the position to bind nucleic acids to solid phases possessing a negatively charged surface or surfaces manifesting a negative charge potential.

From the state of the art, methods and means of purification of nucleic acid are known, with the binding of the nucleic acid taking place to chemically modified solid phases (U.S. Pat. No. 5,523,392; Purification of DNA on Aluminium Silicates and Phosphosilicates; U.S. Pat. No. 5,503,816; Silicates Compounds for DNA Purification; U.S. Pat. No. 5,674,997; DNA purification on modified Siligates; U.S. Pat. No. 5,438,127; DNA Purification by solid phase extraction using a PCl₃ modified glass fiber membrane; U.S. Pat. No. 5,606,046: DNA purification by solid phase extraction using trifluormetric acid washed glass fiber membrane; U.S. patent: DNA purification by solid phase extraction using glass fiber membrane previously treated with trifluoroacetic acid, and then with fluoride ion, hydroxyd ion, or BCL₃; U.S. Pat. No. 5,610,291: Glass fiber membranes modified by treatment with SiCl₃, AlCl₃, or BCl₃ and washing with NaOH to set as a DNA adsorbant; U.S. Pat. No. 5,616,701: DNA purification by solid phase extraction using a hydroxide-washed glass fiber membrane; U.S. Pat. No. 5,650,506. Modified glass fiber membranes useful for DNA purification by solid phase extraction).

The condition for this nucleic acid binding is always the fact that the membranes used for the binding are doted with positive ion charges by chemical modification reactions. Thus, it is obvious that a binding will result between the positively charged surface of the membranes used and the negative ion charge of the phosphate backbone of nucleic acids as a result of Coulomb's interactions. To this extent, the principle of binding of nucleic acids to positively charged solid phases, which is sufficiently known to the experts, is made use of and represents a standard application used for many years, e.g. for DNA/RNA blotting techniques on positively charged nylon filters.

A quite essential disadvantage of these described methods, however, is the fact that they are not suited to nucleic acid isolation i.e. it is completely impossible to isolate nucleic acids from complex starting materials. The starting material is is always a nucleic acid which has already been isolated and, as shown in the U.S. patents quoted, have to be isolated in a way known per se. In particular, one aspect appears unclear to the expert in this context. The binding conditions described (binding under physiological buffer conditions) and elution conditions are identical. It cannot be seen how the nucleic acids are dissolved from the membrane again under the same buffer conditions for the binding of the nucleic acids to the positively charged membrane.

Finally, the means portrayed and the matching methods only possess a very slight practical application. Binding of synthetically produced oligonucleotides to the positive surfaces is also known. This is again done by making use of Coulomb's interaction, i.e. on the basis of the connection of positive and negative charges, e.g. via modified oligonucleotides (connection with amino-linkers or phosphate linkers). These methods also do not enable the isolation of nucleic acids from complex starting materials.

As extensively shown, alternative forms of binding of nucleic acid to membranes with sufficient positive charge for purification exist, albeit not portraying a method for the isolation of nucleic acids. The binding of the nucleic acids is done by Coulomb forces, based on interactions between positive ion charges of the membranes and the negative ion charges of the nucleic acid backbone. This principle therefore appears logically explicable.

On the basis of the isolation of nucleic acids from complex starting materials with anti-chaotropic salts according to the invention, the following was found. It was seen that also negatively charged surfaces or surfaces which can be converted to a negative charging potential are suited for the binding of nucleic acids making use of the lysis/binding buffer systems according to the invention.

The negatively functionalised surfaces or surfaces provided with potentially negative modifications used according to the invention are generated according to methods which are known per se, For example, photochemical coupling of an acetyl group, carboxyl group or hydroxyl group to the surface of a reaction vessel has proven to be suitable.

With the present variant of the method, completely new prospects for a complex nucleic acid analysis are enabled. It was seen that the nucleic acid does not have to have been isolated, as in all the methods already described, for binding of the nucleic acid to negative or potentially negative surfaces. The binding is done from the lysis reaction mixture, i.e. the initial sample containing the nucleic acid is lysed and the nucleic acids released bind to the negatively charged surface (e.g. to a micro-test plate cavity or a reaction vessel).

With the variant of the method according to the invention, totally new single-tube and single-step methods for the isolation of nucleic acids from complex starting materials can be implemented. Such methods offer great advantages for the users in their range of application (simplicity, cheapness, reduction of waste, speed, routine-ability, automation-ability and many more besides).

A further application of this variant of the method entails not only realising extraction of the nucleic acids in a reaction cavity, but also a subsequent target amplification and, if need be, subsequent analysis in the same reaction vessel, if need be performance of hybridisation reactions or allowing sequencing on solid phases to run.

On this basis, for example, a 0.5 ml PCR reaction vessel is modified with a negatively charged or potentially negative functional group by means of techniques known amongst experts. For this, for example, photochemical coupling of an acetyl group, carboxyl group or hydroxyl group to the surface of a reaction vessel is suitable. The sample selected for the isolation of nucleic acid (e.g. full blood) is then put into the reaction vessel, mixed with a lysis buffer containing the anti-chaotropic salt fraction, e.g. ammonium chloride, a detergent and a proteolytic enzyme, and the vessel is incubated for 5 min. at 70° C.

To maximise the binding of the nucleic acid, a detergent/alcohol mixture can be pipetted after the lysis of the starting material. The mixture is then briefly incubated and then poured out of the reaction vessel. The nucleic acid is now bound to the functionalised surface of the reaction vessel and is then briefly rinsed with an alcoholic washing buffer and the alcohol removed by incubation at, for example, 70° C. The elution of the bound nucleic acids is further done by the addition of a low-salt buffer (e.g. 10 mM Tris-HCl) into the reaction vessel and a brief incubation (e.g. 2 min) at e.g. 70° C. The nucleic acid is thus available for subsequent uses.

As shown, all the reactions of the isolation of nucleic acid from a complex starting material take place in one reaction vessel, i.e. lysis of the starting material, binding of the nucleic acids; washing of the bound nucleic acids and elution of the nucleic acids are done in and with a reaction vessel.

The extraction kits of the firm of Qiagen, currently most frequently used world-wide, require one filter cartridge and at least 4 separate reaction vessels for the sequence of lysis, binding, washing and elution, further including multiple centrifugation steps.

On the contrary, the variant of the method according to the invention permits extraction of the nucleic acid without a single centrifugation step, from which an enormous time benefit can be derived. These advantages also relate to the described nucleic acid extraction methods of the quoted U.S. Pat. No. 5,234,809 of Boom.

Alongside possible extraction of nucleic acid, the bound nucleic acid can also remain on the surface of the described 0.5 ml reaction vessel and, e.g., then be used for a PCR application by addition of a complex PCR reaction mixture (primer, nucleotide, polymerase buffer, Taq polymerase, magnesium), i.e. extraction and amplification then take place in the same reaction vessel.

These examples illustrate the enormous advantages and broad applicability to be derived from the invention. In one embodiment, it enables the entire process via amplification and, if applicable, also analysis in, for example, one reaction cavity. With the provision of modified reaction vessels (or also other solid surfaces) and the suitable lysis/binding buffers, this results in new standards in laboratories working in molecular biology and above all in nucleic acid diagnostics, with the well-known problems of sample contamination being drastically reduced as a result of the new potential applicative solutions.

A further advantage and also a further application entails the fact that the surface-fixed nucleic acids are stably fixed on the surface for at least a longer time and are thus also available for later processing, i.e. the PCR reaction does not necessarily have to take place directly after the extraction. A further field of application is fully automated extraction of nucleic acid and, if needed, analysis, making use of the bearing surfaces described here with negative or potentially negative charges, preferably plastic surfaces of suitable reaction cavities (e.g. micro-test plates).

The lysis/binding buffer systems with the anti-chaotropic salts as the main components according to the invention including a proteolytic enzyme if necessary can also be provided as a solid formulation. For this, the mixtures of salts and detergents, additives and, if applicable, enzymes are aliquoted in customary reaction vessels and incubated for a number of hours at 95° C. or lyophilised according to methods known per se and thus transferred to a solid formulation.

These solid formulations in ready-to-use complex reaction mixes for isolation of nucleic acids are long-term storable, i.e. the biological activity of the proteolytic enzyme components remains even in long-term storage (see embodiment). Production of the solid formulation of lysis buffer mixes has been done in this context without addition of protective additives known per se, simply by refrigeration lyophilisation.

All test kits offered commercially for extraction of nucleic acids contain the necessary components individually, certain solutions having to be produced by the user and, over and above this, the solutions having a limited shelf life. A further disadvantage is the fact that the user has to comply with multiple pipetting steps of various individual solutions during isolation of nucleic acids making use of test kits which are currently customary. This dramatically increases the risk of contamination, above all in the area of medical diagnostics. A further disadvantage is the fact that the quantity of starting material is highly limited as a result of any loading limits of customary centrifugation columns in use, which are mainly used for nucleic acid isolation. This is also due to the fact that the lysis and binding buffers necessary for the extraction have to be added to the starting material.

As a result of the provision of a solid formulation as a stable-storage lysis mix on the basis of anti-chaotropic salts, all the existing problems are solved in a quite simple way.

This formulation has the following advantages:
1. long-term storage of ready-to-use lysis buffer mixes,
2. stabilisation of proteolytic enzymes in ready-to-use lysis mixtures and their long-term storage
3. use of larger quantities of starting materials with identical dimensioning of existing centrifugation columns (e.g. trebling the starting quantity)
4. reduction of contamination risks by reducing the pipetting steps and solutions
5. taking of samples in ready-to-use lysis mix also outside the laboratory and possibly long-term storage
6. stable dispatch of samples and cooling

The ready-to-use solid, stable lysis buffer mixes comprising a large number of individual components, including if applicable proteolytic enzymes are simple to handle (also for people without specialist knowledge) as the reaction is simply started by addition of a sample containing the nucleic acid to be isolated. Over and above this, it can be presumed that the mixtures manifest a shelf life of at least 6 months, depending on their ingredients, for which reason transport of the sample at ambient temperature is no longer a problem.

The advantage of solid formulations is based on the fact that, for the lysis of sample materials containing nucleic acids (NAs), a sample containing these NAs is merely placed into the reaction vessel with the long-term storage lysis buffer and the sample is lysed in the reaction vessel in question, possibly by addition of water. Time-consuming and contamination-burdening multiple pipetting steps are no longer necessary at all. Above all for the collection and processing of clinical and forensic samples under field conditions, the known problems are solved by the formulation according to the invention and an easy to handle formulation is available.

Surprisingly, it was then also seen in practical implementation that, following addition of the starting material to be lysed and possibly with addition of a solid sample after addition of H₂O, the solid formulation can be transferred to a liquid phase without any problems tinder standard reaction conditions.

The lysis/binding buffer system can be available as an aqueous solution or as a solid formulation in ready-to-use reaction vessels.

All carrier materials used for isolation by means of chaotropic reagents, preferably glass fibre fleeces, glass membranes, silicon carriers and aerosiles or carrier materials possessing a negatively charged surface or chemically modified surfaces which possess a negative charge potential can act as a solid phase.

The object of the invention is further a method for the isolation of nucleic acids, in particular of DNA, from arbitrary complex starting materials making use of the aforementioned formulations, characterised by lysis of the starting material, binding of the nucleic acids to a carrier material, washing of the nucleic acids bound to the carrier and elution of the nucleic acids.

As a result of the DNA quality achieved, it is also well suited to preparative isolation and purification for DNA for use in genetic therapy.

The object of the invention is also stable-storage, ready-for-use solid formulation of lysis buffer systems for isolation of nucleic acids on the basis of anti-chaotropic salts available as ready-to-use mixes in conventional reaction vessels. The solid formulations of the lysis buffer mixtures are by addition of merely the sample (for liquid samples such as full blood, saliva, cell suspensions, serum, plasma, liquor), for solid starting materials such as tissue, hair roots, blood traces on solid surfaces, cigarette ends, de-paraffinised tissue and many more besides and additional activation by adding water, thus achieving the lysis of the starting material. After the lysis of the starting material, the lysis mixture is incubated in the way known per se, if need be following addition of an ethanolic solution or an alcohol/detergent mixture with the solid phases of any form being used to bind the nucleic acids (suspension, centrifugation column). The subsequent binding of the nucleic acids to the solid phases in question, the washing of the bound nucleic acids and the final elution are done according to the state of the art, as already described.

With these solid formulations, new kinds of solutions result, above all for the fields of application of any form of nucleic acid diagnostics.

We would once more emphasise the fact that the variant of the invention in a single-step method and a single-tube method enables isolation of nucleic acids from complex starting materials, possibly target amplifications and possibly subsequent analysis of the amplified nucleic acid section. The starting material need not be a nucleic acid which has already been isolated, but is the complex starting material containing the nucleic acid. The surface required for the binding of the nucleic acid contains negative or potentially negative functional groups. The binding of the nucleic acid is done in a lysis/binding buffer, the ions needed for the binding of the negatively charged nucleic acid to the negative functionalised surface coming from anti-chaotropic salts.

Thus, the following is possible:
1. Single-tube methods for isolation of nucleic acids from complex starting materials
2. Single-tube methods for isolation of nucleic acids from complex starting materials and subsequent target reproduction
3. Single-tube methods for isolation of nucleic acids from complex starting materials, subsequent target reproduction and subsequent analysis of the reproduced nucleic acid section.

This means both nucleic acid isolation from various starting materials containing DNA, possibly target reproduction and possibly analysis take place in one and the same reaction cavity and possibly on one and the same reaction surface.

The formulations according to the invention and the universal method for binding of nucleic acids to solid phases for isolation, purification and subsequent complex molecular analysis of nucleic acids from arbitrary starting materials and quantities containing nucleic acids mean a new kind of platform technology for the development of integrative fully automatable genetic analysis systems, making it possible to implement sample preparation, target reproduction and target analysis in one reaction cavity.

The invention is now explained in more detail with an example of an embodiment.

Example of an embodiment:

### Binding of DNA to a Solid Phase

1 μg of a DNA length standard (GeneRuler DNA Ladder Mix, Fermentas) was transferred to a centrifugation column with a glass membrane in a buffer comprising components shown in the illustration (Micro Spin Säule, Safeclick). There followed a centrifugation for 2 min at 12,000 rpm and rejection of the filtrate. After drying by a short centrifugation step (12,000 rpm for 2 min), 10 μl of an elution buffer (10 mM Tris-HCl; pH 8.0) was added, followed by elution of the DNA by centrifugation for 1 min at 10,000 rpm.

10 μl of the eluted DNA was then placed onto an agarose gel and portrayed after dyeing with ethidium bromide (FIG. 1).

1. Formulations without chaotropic components for the isolation of nucleic acids with binding to a solid phase, in particular of DNA from arbitrary complex starting materials, containing
a lysis/binding buffer system manifesting at least one anti-chaotropic salt component,
a solid phase,
washing and elution buffers which are known per se,
wherein the concentration of the anti-chaotropic salt components is between 0.001 mM and 0.1 M, preferably 0.1 mM.

2. Formulations according to claim 1, wherein
the anti-chaotropic salt component is an ammonium, caesium, sodium and/or potassium salt, preferably ammonium chloride.

3. Formulations according to claim 1 or 2, wherein
the lysis/binding buffer system manifests detergents and, if applicable, Additives.

4. Formulations according to claim 3, wherein
detergents and additives are Tris-HCl, EDTA, polyvinyl pyrrolidone, CTAB, TritonX-100, N-lauryl-sarcosine, sodium citrate, DTT, SDS and/or Tween.

5. Formulations according to one of the claims 1 to 4, wherein the lysis/binding buffer system manifests an alcohol for binding onto the solid phase.

6. Formulations according to one of the claims 1 to 5, wherein the lysis/binding buffer system manifests enzymes, preferably protein-decomposing enzymes.

7. Formulations according to one of the claims 1 to 6, wherein the lysis/binding buffer system is available as an aqueous solution.

8. Formulations according to one of the claims 1 to 6, wherein the lysis/binding buffer system is available as a solid, storage-stable formulation in ready-to-use reaction vessels.

9. Formulations according to one of the claims 1 to 8, wherein all carrier materials applied for isolation by means of chaotropic reagents function as the solid phase, preferably glass fibre fleeces, glass membranes, glasses, zeoliths, silicone carriers.

10. Formulations according to one of the claims 1 to 8, wherein carrier materials possessing a negatively functionalised surface or manifesting functionalised surfaces which can be converted to a negative charging potential function as a solid phase.

11. Formulations according to claim 10, wherein
the surface of the carrier material has been modified with an acetyl group, carboxyl group or hydroxyl group.

12. Method for the isolation of nucleic acids, in particular of DNA, from arbitrary complex starting materials making use of formulations according to one of the claims 1 to 9, wherein the starting material is lysed, the binding of the nucleic acids to a solid phase takes place, the nucleic acids bound to the carrier are washed and the elution of the nucleic acids takes place.

13. Method for the isolation of nucleic acids according to claim 12, wherein the material containing the DNA
is brought into contact with a lysis/binding buffer system entailing an aqueous solution containing an anti-chaotropic salt component, at least one detergent, if need be additives and if need be a proteolytic enzyme, and
with a solid phase, if need be making use of an alcohol,
is then washed and the nucleic acid dissolved from the solid phase.

14. Method according to claim 13, wherein starting materials are compact plant materials such as fruits, seeds, leaves, needles etch, clinically relevant samples such as full blood, tissue, micro-bioptates, paraffinised materials, ercp samples, swab material from smears, foodstuffs such as fish, cooked meats, preserves, milk, forensic samples such as hair roots, cigarette ends, blood traces and other samples containing DNA.

15. Method for the isolation of nucleic acids, in particular of DNA, from arbitrary complex starting materials making use of formulations according to one of the according to one of the claims 1 to 8 and 10 to 11, wherein
the starting material is brought into contact with a negatively functionalised surface or with a surface which has been chemically modified in such a way that it can be converted into a negative charge potential in a single-tube or a single-step process and is lysed,
the nucleic acid is bound to this surface,
the bound nucleic acid is washed and eluted if necessary.

16. Method according to claim 16, wherein negatively functionalised surfaces are correspondingly modified planar surfaces, filter membranes, conventional plastic vessels or micro-test plates.

17. Method according to claim 16 or 17, wherein the nucleic acid is subsequently subjected to an amplification reaction in the same reaction mixture and then, if need be, an analysis of the genetic sequences is carried out.

18. Method according to claim 16 or 17, wherein the nucleic acid is then hybridised or sequenced in the same reaction mixture.

19. Use of anti-chaotropic components in a lysis/binding buffer system according to claim 1 for isolation and purification of nucleic acids with binding to a solid phase.

20. Use according to claim 19, wherein anti-chaotropic salt components are ammonium, caesium, sodium and/or potassium salts, preferably ammonium chloride.

21. Use according to one of the claims 19 to 20, wherein the lysis/binding buffer system is used as an aqueous solution.

22. Use according to one of the claims 19 to 21, wherein the lysis/binding buffer system is available as a solid, storage-stable formulation.

23. Use according to one of the claims 19 to 22 for preparative isolation and purification for DNA for use in genetic therapy.
